(19) United States
(12) Patent Application Publication
(10) Pub. No.: US 2021/0244393 A1
(43) Pub. Date: Aug. 12, 2021

(54) STEERABLE CONDUIT FOR TRANSSEPTAL PASSAGE OF DEVICES TO THE AORTA

(71) Applicant: Synecor LLC, Durham, NC (US)

(72) Inventors: Richard S. Stack, Chapel Hill, NC (US); Kevin W. Johnson, Durham, NC (US); William L. Athas, Chapel Hill, NC (US); Emer M. FEERICK, Galway (IE)

(21) Appl. No.: 17/170,802

(22) Filed: Feb. 8, 2021

Related U.S. Application Data
(60) Provisional application No. 62/971,907, filed on Feb. 7, 2020.

(51) Int. Cl. A61B 17/00

(52) U.S. Cl. CPC A61B 17/00234; A61B 2017/3425; A61B 2017/00323; A61B 2017/00243

(57) ABSTRACT

A conduit for creating a passage from a right atrium to a left atrium, through a mitral valve into the left ventricle, and to provide a passage from the left ventricle into the aortic valve. The conduit includes an elongate tubular member having a shaft with a proximal section and a distal loop section at a distal end of the proximal section. The distal loop section includes a passive proximal curve, a steerable distal curve, a generally straight segment extending between the curves, and a distal tip. The shaft in the distal loop section is steerable to cause it to curve back on itself so that proximal curve is formed by a part of the shaft that is closer along the length of the shaft to the distal tip. The shapes of the proximal and distal curves are selected to direct the distal tip into the mitral valve after it has crossed the inter-atrial septum from the right atrium to the left atrium of the heart, and to orient the distal opening of the distal tip towards the aortic valve when the proximal curve is in the mitral valve and the distal tip is in the left ventricle.

This application claims the benefit of U.S. Provisional Application No. 62/971,907, filed Feb. 7, 2020, which is incorporated hereby reference.

### BACKGROUND

Various medical procedures in use today involve passage of devices from the right side of the heart to the left side across the inter-atrial septum in a well-established technique known as transseptal catheterization.

Commonly owned application Ser. No. 16/578,375, Systems and Methods for Transseptal Delivery of Percutaneous Ventricular Assist Devices and Other Non-Guidewire Based Transvascular Therapeutic Devices, filed Sep. 22, 2019 (Attorney Ref: SYNC-5000R), which is incorporated herein by reference, discloses a system and method for delivering therapeutic devices positionable at the aortic valve, and gives as a primary example its use to deliver pVADs. In that application, transseptal catheterization is used to deliver a long flexible cable such that it extends from the venous vasculature through the heart to the arterial vasculature. Once positioned the cable has one end extending from the right subclavian vein and an opposite end extending from the right or left femoral artery. Once positioned in this way, a grasper is attached to the cable at the femoral artery, and the cable is withdrawn from the right subclavian vein to position the grasper along the route previously occupied by the cable. The grasper is then attached at the right subclavian vein to a pVAD and pulled from the femoral artery while the pVAD is simultaneously pushed at the right subclavian vein. This combination of pulling and pushing force moves the pVAD into the heart, across the septum and the mitral valves, and into its final position at the aortic valve.

Commonly owned co-pending application PCT/US2017/62913, filed Nov. 22, 2017, published as WO/2018/098210 (incorporated herein by reference) discloses a system and method for delivering mitral valve therapeutic devices to the heart (such as devices for positioning a replacement mitral valve or devices for treating a native mitral valve) using a transseptal approach. In that application, transseptal catheterization is used to position a cable that is used to deliver a therapeutic device to the mitral valve site. Once the cable is positioned it has one end extending from the right femoral vein and an opposite end extending from the left or right femoral artery. The mitral valve therapeutic device is attached to the cable at the right femoral vein. The cable is then pulled at the femoral artery while the mitral valve therapeutic device is simultaneously pushed at the right femoral vein. This combination of pulling and pushing force moves the mitral valve therapeutic device into the heart, across the septum and to its final position at the mitral valve.

Co-pending and commonly owned application Ser. No. 16/860,015, filed Apr. 27, 2020 and entitled Transseptal Delivery System and Methods for Therapeutic Devices of the Aortic Valve (incorporated herein by reference) describes for delivering an aortic valve therapeutic device, such as a TAVR delivery system carrying a TAVR valve, to an aortic valve site using a modified approach to the aortic valve site using a system that is similar to that described in U.S. application Ser. No. 16/578,375. In that application, the therapeutic device is introduced into the vasculature on the arterial side (e.g., via the right femoral artery “RFA”) vs the venous side as described in each of the co-pending applications. The system and method described in that application allows the TAVR delivery system to be precisely maneuvered coaxially into the center of the native or a prosthetic aortic valve, orthogonal to the aortic valve annulus and away from the sub-valvular conduction system.

In each of the above procedures, a Brockenbrough type of transseptal catheterization is initially performed using access from the right femoral vein, and then other devices make use of the transseptal access created to aid in positioning of the wire or cable that is to ultimately reach the aorta and femoral artery. A common challenge of these procedures is the need to provide safe passage for such devices downwardly within the left atrium from the transseptal puncture site towards the mitral valve, and then through the mitral valve and upwardly within the left ventricle to the aortic valve, without engaging the delicate chordae tendineae of the mitral valve, and then into the aorta beyond the level of the coronary sinuses to the aortic arch and descending aorta. Above-referenced application Ser. No. 16/578,375 describes a right to left conduit (RLC) configured to navigate this passage, while possessing material properties that resist kinking and transmit the torque needed to achieve delivery with minimal impact to the chordae or endocardial tissue.

The present application describes a modified RLC incorporating a steerable portion.

### BRIEF DESCRIPTION OF THE DRAWINGS

FIG. 1A is a side elevation view of a Right-to-Left conduit (“RLC”).

FIG. 1B is a side elevation view of the distal end of the RLC, with the distal curve articulated to its curved orientation, and with the remaining portion of the shaft lying in a straight configuration.

FIG. 2A is a side elevation view of the distal part of the RLC.

FIG. 2B is a partially cut-away view of the region of the RLC encircled in FIG. 2A.

FIGS. 3A through 4 are a series of figures schematically illustrating steps in which the RLC is used to help deliver a cable device that is to be passed through the heart between the venous and arterial vasculature, in which:

FIG. 3A illustrates positioning of the RLC following transseptal advancement of a wire through a Brockenbrough transseptal catheter and into the left atrium, and subsequent movement of the RLC over the wire across the septum.

FIG. 3B illustrates positioning of the RLC after its distal tip has passed through the mitral valve into the left ventricle.

FIG. 4 illustrates the position of the RLC in the left ventricle oriented towards the aortic valve. The arrows in FIG. 4 represent the “windshield wiper” motion of the distal tip of the RLC after it passes through the mitral valve but before the wire is advanced through it into the aortic valve.

### DETAILED DESCRIPTION

The present application describes a Right-to-Left conduit 100 (“RLC”) RLC having similar properties to that described in the Background and in commonly-owned and co-pending U.S. application Ser. No. 16/578,375, but that has been modified to allow a user to actively steer it to a select trajectory for rapid access to the aortic root and left ventricle outflow tract from the trans-septal position, while maintaining a highly flexible structure and uncompromised torque response.

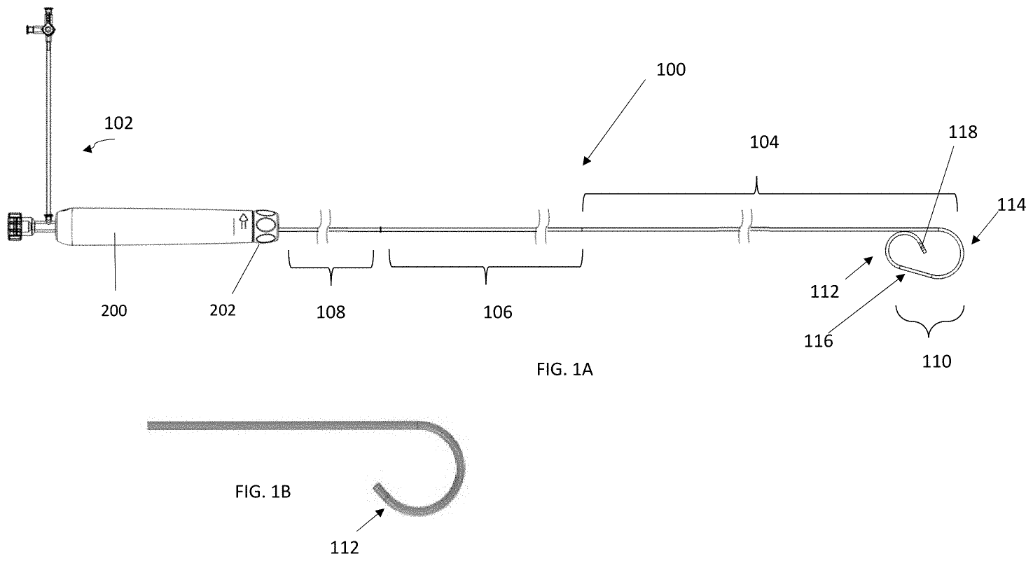

Referring to FIG. 1A the Right-to-Left conduit 100 (“RLC”) is an elongate tubular catheter having a length sufficient to permit it to extend from the RFV of a human adult to the right atrium, across the interatrial septum (via a trans-septal puncture) to the left atrium, through the mitral valve, left ventricle, aortic valve to the aortic arch, and then to the descending aorta. In a preferred embodiment, this length exceeds 150 cm, and it may be 160 cm or longer. A lumen extends through the RLC 100 from a proximal port 102 to an opening at the distal end. A flush port is also fluidly connected with the lumen of the RLC as shown.

The RLC has a distal portion 104, an intermediate portion 106, and a proximal portion 108. The proximal and intermediate portions, 108, 106 and much of the distal portion 104, are of generally straight tubular construction. These parts of the shaft may be collectively referred to as the main body of the shaft. The distal portion 104 includes a distal loop 110 that has been shape set. The shape of the loop helps the distal end of the RCL pass into the mitral valve after it has crossed the intra-atrial septum from the right to the left side of the heart, further aids in orienting the distal opening of the RLC towards the aortic valve (as will be discussed in connection with FIG. 4) when the distal part of the RLC is in the left ventricle.

More particularly, the distal loop 110 includes a distal (where for the purposes of this description of the curves of the RLC the term “distal” and “proximal” are used in regard to the entire length of the catheter) curve in regard to the entire length of the catheter 112, a more proximal curve 114, a generally straight segment 116 extending between the curves, and a distal tip 118. The RLC is shape set with the longitudinal axes of the distal and proximal curves in a common plane, although in alternative embodiments they might lie in different planes. In other embodiments, one or both of the curves might be formed with a shape where the longitudinal axis forms a three-dimensional shape and thus does not lie within a single plane. The generally straight segment 116 may be straight or it may be curved with a very large radius of curvature to produce a significantly more gradual curve than the proximal and distal curves.

The curves 112, 114 are arranged to cause the distal loop 110 to curve back on itself, so that the distal curve 112 is formed by a part of the RLC shaft that is closer along the length of the shaft to the distal tip 118 than is the proximal curve 114. The radius of the distal curve is smaller than that of the proximal curve, so that the lateral width (perpendicular to the longitudinal axis of the straight section of the shaft) of the loop 110 tapers inwardly from a proximal to distal direction. The distal tip is preferably enclosed within the loop, bounded by distal and proximal curves, segment 116, and the main body of the shaft. It is also, preferably, oriented with its distal opening facing away from the main body of the shaft.

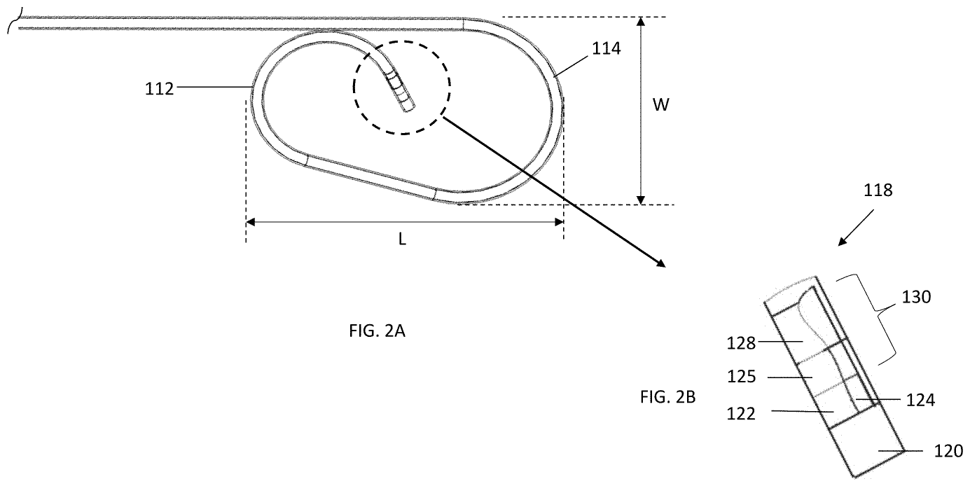

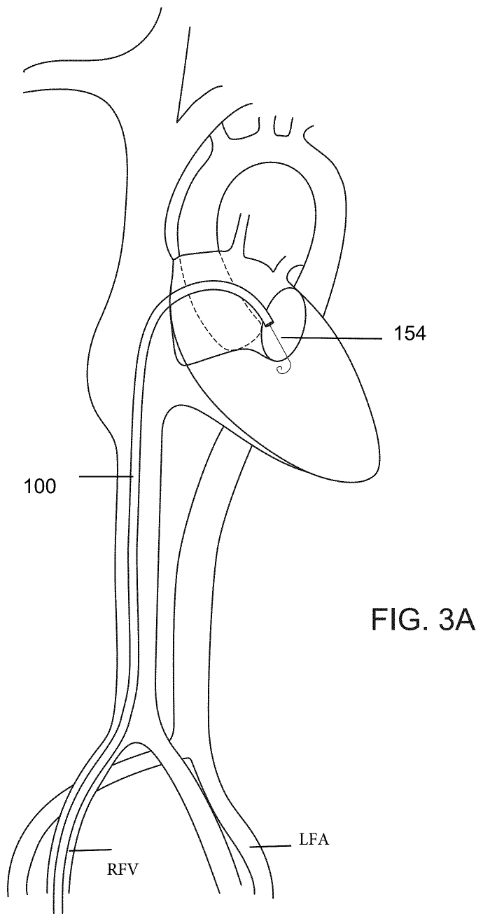

Referring to FIG. 2A, the radii of the distal and proximal curves, the length of the generally straight segment 116 along its longitudinal axis, the widest lateral dimension W of the distal loop (measured in a direction perpendicular to the longitudinal axis of the straight part of the RLC), and the longitudinal length L of the distal loop (in a direction parallel to the longitudinal axis of the straight part of the RLC) are proportioned so that when the proximal curve 114 is within mitral valve, the distal curve 112 is positioned in the left ventricular outflow tract (as shown in 3B) and the tip 118 is oriented towards, and in close proximity to, the aortic valve. In one embodiment, length L may be in the range of 65-95 mm, with a preferred range of approximately 70-90 mm, or more preferably approximately 75-85 mm. Width W may be in the range of 35-65 mm, with a preferred range of approximately 40-60 mm, or more preferably approximately 45-55 mm. The radius of the distal curve 112 may be in the range of 5-35, with a preferred range of 10-30 mm, and a most preferred range of 15-25 mm. The radius of the proximal curve 114 may be in the range of 10-40 mm, with a preferred range of 15-35 mm, and a most preferred range of 20-30 mm.

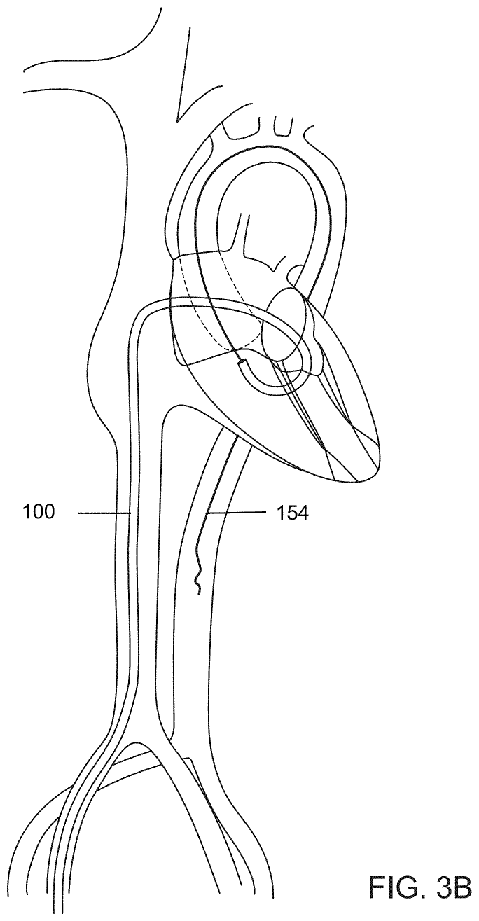

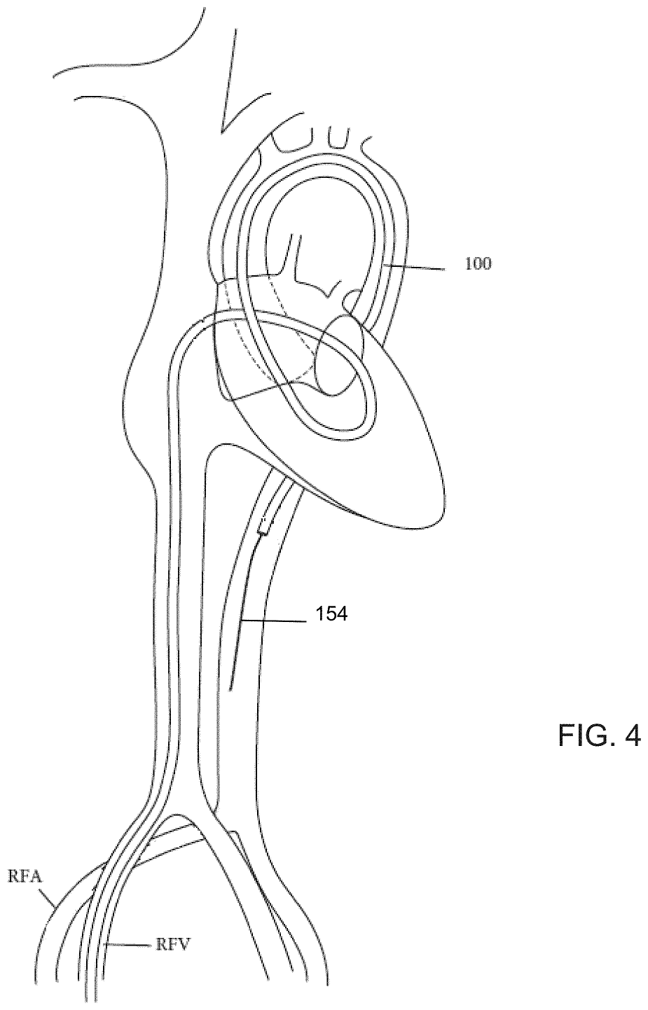

In the embodiment that is shown, the widest lateral dimension of the distal curve 114, taken in a direction perpendicular to the longitudinal axis of the main shaft of the conduit, is wider than the widest lateral dimension of the proximal curve 112 taken in a direction perpendicular to the longitudinal axis of the main shaft of the conduit. However, in other embodiments these widths may be approximately equal but the curvature would be ideally selected to orient the distal tip 118 towards the interior of the loop, thus ensuring that when the RLC is positioned with its distal tip in the left ventricle, the tip is generally oriented towards the aorta as shown in FIG. 3B.

The circumference of the curve 112 passes closely adjacent to the straight section of the main body of the main shaft in distal region 104, so that the main body extends tangentially with respect to the circumference of the proximal curve 114. The curvature of the proximal curve continues beyond this tangential area, so that the distal tip 118 is disposed within a generally enclosed loop as noted above. In other embodiments, the proximal curve and/or the distal tip may cross the straight section of the shaft.

The RLC is constructed for active steering of the distal curve 112, preferably by more than 180 degrees in a single direction as illustrated in FIG. 1B. In one embodiment, steering of the distal curve is effected by increasing tension on a pull element (which may be a wire, cable, filament secured at the RLC's tip) using an actuator 202 in the RLC's handle 200. While in some embodiments a return wire may be used to return the distal curve 112 to the straight configuration, in this embodiment the flexible shaft returns itself to the generally straight configuration when tension on the pull element is eased or released. Note that while the RLC is described as being shape set, in some embodiments the distal curve 112 is not shape set, and steering is relied on to move it to the desired shape during use.

In an alternative embodiment, the RLC includes one or more additional pull elements that may be tensioned to effect steering of the proximal curve 114. However, in the present embodiment, changes to the proximal curve 114 during use are driven using a guidewire extending through it, as is described in the Method section below, rather than using a pull element for active steering.

The materials for the RLC are selected to give the conduit sufficient column strength to be pushed through the vasculature, torqued to orient its tip towards the aortic valve, and tracked over a wire, and it should have properties that prevent the distal loop 110 from permanently deforming as it is tracked over a wire. Although the distal loop 110 is moved out of its pre-shaped loop configuration to track over the wire, it is important that the shape-setting of the curves be retained. Otherwise, the performance benefits of the distal loop's shape which, as evident from the Method description below are to aid proper movement into and through the mitral valve, to orient the tip of the RLC towards the aortic valve, and to track over the wire all the way to the descending aorta will not be realized.

Preferred material properties for the RLC will next be given, although materials having different properties may be used without departing from the scope of the invention. The shaft includes an outer jacket formed suitable polymeric material (e.g., polyether block amide, “PEBA,” such as that sold under the brand name Pebax). A wire braid extends through shaft portions 108, 106 and most of 104 to enhance the torqueability of the RLC. A lubricious liner made using PTFE, ultra-high molecular weight polyethylene (UHMWPE), or like material also extends through these sections, allowing smooth relative movement between the RLC and the wire and cable that pass through it. The braid and liner terminate in the distal tip 118 as will be described with respect to FIG. 2B. The liner, braid and outer jacket are preferably subjected to a reflow process to create a composite material.

The most proximal portion 108 of the RLC, which may be between 450 and 550 mm in length (most preferably between 485 and 525 mm), is preferably formed from a relatively stiff material made from, as one example, 72D Pebax. Adjacent to the proximal portion 108 is the intermediate portion 106. This portion may have a length between 500-600 mm (most preferably between 530-570 mm), and it is preferably formed of fairly stiff material, but one that is more flexible than that used for the most proximal portion. As one example, this material may be 55D Pebax. These materials give the proximal and intermediate portions 108, 106 sufficient column strength and torqueability needed for its intended use.

Shaft section 104 is designed to be more flexible that the more proximal sections, because it must be able to pass through the heart during use. This section may be formed of a material such as 40D Pebax, although it is more preferably formed of a blend of 40D and 55D Pebax. This avoids an abrupt transition at the junction between sections 104 and 106 and can help to avoid kinking at that junction. The ratio of 40D to 55D material in the blend may be 50:50 or an alternative ratio. Shaft section 104 makes up the most distal part of the straight section of the main shaft, as well as both the proximal curve 114 and the segment 116. Directly adjacent to the section 104 is a short section of soft durometer material (e.g. Pellethane 80A or Pebax 25D) in the distal curve 112. This use of materials allows for active steering of the distal curve 112, while retaining greater stiffness just proximal to the distal curve to permit the more proximal part of the loop 110 to follow the anatomy during advancement without buckling. The length of shaft section 104 plus the distal curve 112 is preferably between 510 and 610 mm, and more preferably between 540 and 580 mm.

A preferred configuration for the distal tip 118 will next be described. Referring to FIG. 2B, which is partially cut away to show features below the outer extrusion, the distal tip 118 includes an atraumatic distalmost section 120 formed of soft 35D Pebax or similarly soft material. Just proximal to the distal most section is a more rigid section (e.g. 55D Pebax) 122, which includes a radiopaque marker band 124 (e.g. Ptlr) and the distal-most part of the lubricious liner (not shown). In the next most proximal section 130 is the pull ring 125, to which the pull element is fixed, and the terminal portion of the braid 128. These are covered by a more rigid material such as 72D polyethylene or similar material. Each of the sections 120, 122, 130 is very short in length, and preferably between 2-6 mm. As shown, the distal tip is preferably a generally straight section of the RLC extending from the distal curve 112.

It should be pointed out that while a number of preferred features for the RLC have been described above, alternative embodiments of the RLC might use any sub-combination of the above-described features alone or with other features not described here.

### Method of Use

A method of placing the RLC via transseptal catheterization will next be described. The purpose of RLC placement is to position a conduit extending into a femoral vein and across the heart via the interatrial septum, through the mitral valve into the left ventricle, and then oriented towards the aortic valve. The RLC is then advanced through the aortic valve, beyond the coronary sinuses and through the ascending and descending aorta. In that position it enables a user to deploy an arterio-venous cable in the descending aorta that can be used to deliver other devices into the heart in procedures such as those discussed in the Background section of this application.

As an initial step, the practitioner obtains percutaneous access to the vessels that are to be used for the intravascular procedure. For the purposes of this discussion, it will be assumed that access to the right and or left femoral artery (RFA, LFA), the right or left femoral vein (RFV, LFV), and, if the procedure is one involving advancement of devices from a superior location (as discussed in the Background), the right subclavian vein (RSV) or the left subclavian vein (LSV), or the right or left internal jugular vein (RIJV, LIJV). One such sheath is shown in FIG. 3, positioned in the RSV.

A Brockenbrough transseptal catheter (BTC) is introduced through the RFV and, using the well-known technique of transseptal catheterization, is passed from the right atrium (RA) into the left atrium (LA). A wire 154, which may be an 0.035″ wire such as the Abbott Versacore wire, is passed through the BTC and into the left atrium (LA).

The BTC is withdrawn at the RFV and exchanged for the RLC 100, which is advanced over the wire 154. The RLC preferably has been filled with an 80/20 saline-contrast solution for additional visibility under fluoroscopy. After it has crossed the inter-atrial septum into the LA, the RLC is advanced toward the lateral edge of the LA. From this position the wire is withdrawn proximally into the RLC (proximal to the loop 110, labeled in FIGS. 1 and 2A). The RLC is rotated counterclockwise about the axis of the main body portion as the wire is slowly withdrawn. This causes the tip to drop in an inferior direction into and through the mitral valve MV towards the left ventricle LV. Once the tip is through the MV, the RLC continues to be advanced, its shape and active steering using the pull element causing the distal end of the tip to move in a right-ward (the patient's right) and anterior direction. This direction of motion is needed to orient the tip 118 towards the aortic valve AV, since the aortic valve is anterior and to the right of the mitral valve.

The RLC's curvature as well as active steering of the distal end directs its tip towards the aortic valve. FIG. 3B shows the distal tip of the RLC pointed towards the aortic valve. As shown, the RLC extends within the inferior vena cava, extends through the interatrial septum (not shown), drops into the mitral valve and forward into the left ventricle.

It should also be mentioned that movement of the RLC through the heart as described above is optimally performed while selectively using a variable stiffness guidewire through the RLC, allowing the variations in curvature and stiffness along the length of the RLC to work together with the different degrees of regional stiffness of the guidewire. This is particularly useful to direct the shape of the proximal curve 114 which, in this embodiment, is not configured to be actively steered by pull elements. One useful type of variable stiffness guidewire is one having at least three segments of different flexibility. The first, and most distal of those segments has the greatest flexibility. A second segment is proximal to the distal segment and has less flexibility than the first segment, and a third segment is proximal to, and less flexible than, the second segment. In one specific example, the first and third segments are directly adjacent to the second segment.

Where a variable stiffness guidewire is used, during the step of crossing the septum with the RCL, the stiffest segment of the guidewire is positioned through curves 112, 114 of the RLC, forming it into a gently curved configuration. In this more straightened configuration, advancement of the RLC, after it crosses the septum, causes its tip to cross the left atrium to a position beyond the mitral valve, and optionally in a left pulmonary vein. After the RLC reaches this position, the guidewire is withdrawn so the most flexible distal section, at least within the curve 112 of the RLC, causing the RLC to return to a more curved orientation due to the withdrawal of the stiff part of the guidewire from the loop 210 of the RLC. Counterclockwise torque is then applied as the RLC is withdrawn, causing the RLC tip to move anteriorly through the mitral valve. The tip will drop from the mitral valve into the left ventricle. The RLC is pushed with clockwise torque, or with alternating clockwise and counterclockwise torque, while the RLC is actively steered at the distal curve 112 (by manipulating the actuator 202 to tension the pull element). This directs the RLC tip adjacent to the ventricular septum and pointing to the left ventricular outflow tract.

When the distal tip 118 of the RLC 100 positioned in the LV, its curvature directs its tip towards the aortic valve as shown in FIG. 4. With the RCL positioned in this way, the guide wire 134 is advanced through the aortic valve, around the aortic arch, and into the descending aorta, allowing the RLC to be advanced to the descending aorta on the stiffer segment of the guidewire.

Before the method proceeds, one of various methods, including those described in the prior referenced applications, may be performed to confirm that the wire path is free of chordae entrapment at the mitral valve. The RLC 100 is then advanced to the descending aorta.

The subsequent steps from this point may differ depending on the procedure that is to be performed. For example, some procedures may involve placement of a cable to extend between the venous and arterial vasculature as described in the applications referenced in the Background section. This may be performed by replacing the wire in the RLC with the cable from the venous side until it extends from the RLC in the aorta, and then advancing a snare from the right femoral artery (RFA) towards the descending aorta to engage the cable. The snare is exteriorized from the RFA to draw the end of the cable that is proximal to the RFA out the RFA. At this point the cable extends between the RFV and the RVA (although it should be understood that the left femoral vein and/or artery might instead be accessed in place of these right side vessels).

The steps that happen next are dependent on whether the end of the cable that is on the venous side needs to be access from a superior site or a femoral site. If a procedure to deliver a mitral valve therapeutic device, such as that described in PCT application WO/2018/098210, is to be carried out, the subsequent steps are performed using the cable extending between the femoral vein and femoral artery (e.g. the RFA and RFV as shown). A similar cable arrangement is used for the TAVR procedure described in U.S. Ser. No. 16/860,015. If a procedure to deliver a pVAD is to be carried out, the venous end of the cable may be exteriorized from the RSV using steps described in Commonly owned application Ser. No. ______, Systems and Methods for Transseptal Delivery of Percutaneous Ventricular Assist Devices and Other Non-Guidewire Based Transvascular Therapeutic Devices, (Attorney Ref: SYNC-5000R). Naturally, other applications may require different steps, e.g. having the RLC itself extend exteriorly from both the RFV and the RFA.

All patents and patent applications referred to herein, including for purposes of priority, are fully incorporated herein by reference.

We claim:

1. A conduit for creating a passage from a right atrium to a left atrium, through a mitral valve into the left ventricle, the conduit comprising:
an elongate tubular member having a shaft with a proximal section and a distal loop section at a distal end of the proximal section, wherein in the distal loop section includes a proximal curve section, a distal curve section, a generally straight segment extending between the proximal and distal curve sections, and a distal tip;
a handle with an actuator;
a pull element anchored at the distal tip and extending through the tubular member from the distal tip to the actuator, the actuator moveable from a first position to a second position to increase the tension on the pull element, wherein when the actuator is in the first position the distal curve section assumes a generally straight configuration, and wherein when the actuator is in the second position the pull element pulls the distal curve section to a distal curve of at least 180 degrees.

2. The conduit of claim 1, wherein the proximal curve section is shape set to passively form a proximal curve.

3. The conduit of claim 2, wherein the shapes of the proximal and distal curves are selected to direct the distal tip into the mitral valve after it has crossed the intra-atrial septum from the right atrium to the left atrium of the heart, and to orient the distal opening of the distal tip towards the aortic valve when the straight segment or the distal curve is in the mitral valve and the distal tip is in the left ventricle.

4. The conduit of claim 1, wherein the proximal curve has smaller radius than distal curve, so that distal loop has a width that tapers from a distal to a proximal direction.

5. The conduit of claim 1, wherein the proximal curve portion loop is formed using a blend of 40D and 55D durometer polymeric material and the distal curve portion is formed of a Shore 80A polymeric material.

6. The conduit of claim 5, wherein the shaft includes a first portion formed using a polymeric material of 72D durometer, and a second portion distally adjacent to the first portion formed using a polymeric material of durometer of 55D, the proximal curve portion distally adjacent to the second portion.

7. The conduit of claim 1, wherein the conduit is of sufficient length to extend from a femoral vein and positionable transseptally from the right atrium to the left atrium;
wherein the shapes of the proximal and distal curves are selected to direct the distal tip into the mitral valve when the proximal section is pushed from the femoral vein after the distal tip has crossed the intra-atrial septum from the right atrium to the left atrium, and to cause the distal opening of the distal tip to be actively steerable to an orientation in the left ventricle facing the aortic valve.
